(19) United States
(12) Patent Application Publication
(10) Pub. No.: US 2009/0192529 A1
(43) Pub. Date: Jul. 30, 2009

(54) SOFT TISSUE REATTACHMENT MECHANISM

(76) Inventor: Michael Kaveney, (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/357,884

(22) Filed: Jan. 22, 2009

Related U.S. Application Data
(60) Provisional application No. 61/022,637, filed on Jan. 22, 2008.

(51) Int. Cl. A61B 17/08

(52) U.S. Cl. 606/151

(57) ABSTRACT

Among other things, apparatus and methods for reattaching soft tissue to bone are disclosed. In one embodiment, a monolithic anchor having a head flexibly attached to a stem including ridges or grooves is provided. A locking member may be placed over the stem, which has one or more pawls to engage the ridges or grooves to inhibit the locking member from coming off of the stem. A tube may be provided for access to the surgical location. With the tube placed through soft tissue and cortical bone or other access, the anchor with the head pivoted to be parallel with or along the stem may be placed through holes in the soft tissue and cortical bone. The head is turned transverse to the stem, preventing the head from passing back through the cortical bone. The locking member is placed over the stem to hold the soft tissue to the bone.

The present application claims the benefit of U.S. Provisional Application Ser. No. 61/022,637, filed on Jan. 22, 2008, the entirety of which is incorporated by reference herein.

The present disclosure is directed to devices and methods for reattaching soft tissue to bone. In particular, the disclosure includes devices that quickly, easily and with less trauma to the patient hold soft tissue to bone so that healing can occur, and methods for implanting the same.

### BACKGROUND

In the field of orthopedic repair of soft tissue/bone connections, techniques using sutures to hold the soft tissue to the bone have been used for many years. For example, in repairing soft tissue tears in the rotator cuff, tissues such as the rotator cuff tendon are tied to the superior part of the humerus (head, tubercle or other part) using sutures. To do so, the orthopedic surgeon must perform an open surgical procedure, creating an incision through dermal layers, muscle, and other tissue to reach the superior part of the humerus. The surgeon introduces one or more sutures through and/or around the bone and ligament and ties the suture(s) into a knot to hold the bone and ligament together. Frequently, however, the surgical space or conditions make tying one or more knots difficult, with the result that the knots may not be in the right place along the suture and the suture is too slack or too tight. A suture that is too slack may not hold the tissues together properly, or may permit slippage between them, while a suture that is too tight may damage the ligament or associated tissues. Further, in some cases the knot(s) may not be entirely secure, resulting in slippage or in release of the suture(s).

In attempting to solve the problems associated with knotting sutures, the art has attempted to provide other ways to hold sutures in a configuration that will be relatively easy to use and effective in holding soft tissue. Such examples include applying energy to the knot or suture to fuse the suture, or adding a clamp or other gripping mechanism to hold sutures together. These features can be effective in limiting some degree of slippage, but they do not address fundamental difficulties of using sutures in such soft tissue reattachments, including the requirement of open surgery and the need for high dexterity in placing and configuring the sutures.

Consequently, a need has existed for some time for apparatus and methods to connect soft tissue to bone that does not require sutures for a secure attachment.

### SUMMARY OF THE INVENTION

The present disclosure is directed to, among other things, a non-suture apparatus for attaching soft tissue to bone. Such an apparatus can include an anchor which may have a monolithic head, stem, and neck. The head may be substantially inflexible with an underside directly connected to the neck. The stem may be elongated with a central axis, a first end that is directly connected to the neck and a second opposing end that is tapered, and a series of ridges that are substantially perpendicular to and offset from the central axis found at least on a portion of the stem adjacent to the neck. The neck may have at least a portion that is flexible and elastic, wherein the anchor has a first unstressed configuration in which the head is transverse to the stem, and the flexible and elastic neck is bendable so that the head can be subjected to stress and moved to a second configuration in which the head is substantially parallel to the stem. The elasticity of the neck allows the anchor to change to substantially the first configuration on the removal of the stress. A lock is placed on the stem, which lock may have an upper surface generally facing away from the head, a lower surface generally facing toward the head, and a central hole between the surfaces defined by an internal wall. The central hole may have a width larger than a width of the stem, and at least one pawl extends from the internal wall in a direction generally toward the upper surface of the lock. The pawl(s) are of a length to be able to interengage with the ridges on the stem, so that the lock can easily slide over the ridges on said stem toward the head, and the pawl(s) prevent the lock sliding over the ridges on the stem away from the head.

Among the methods disclosed herein is a method of minimally-invasively performing soft tissue reattachment procedures, such as rotator cuff repair surgery. Minimally-invasive holes can be created through detached soft tissue and through the cortical bone of the humerus so that the holes align when the soft tissue is on the humerus in a desired relationship. A narrow tube is inserted through the holes, so that access through the tube to a point below the cortical bone is gained. An anchor (e.g. a monolithic anchor) is placed through the tube and the holes, the anchor having a stem with ridges, a head having a width larger than a width of the holes, and a flexible and elastic neck directly joining the stem and head. The neck may be bent so that the head is substantially parallel to a portion of the stem when the anchor is within the tube, and wherein the elasticity of the neck causes the head to move with respect to the stem when the head has cleared the hole in the cortical bone of the humerus, so that the head is transverse to the stem and abuts cortical bone of the humerus from inside the humerus, preventing the anchor from being pulled through the holes. A lock can be placed on the stem, the lock having a central hole and at least one pawl configured to interengage with the ridges of the stem so that the lock can be moved toward the head over the ridges, and that the lock cannot be moved away from the head over the ridges due to interaction between the at least one pawl and one or more of the ridges. The lock is moved along the stem so that the soft tissue and the cortical bone of the humerus are pressed between the head and the lock.

### BRIEF DESCRIPTION OF THE DRAWINGS

FIG. 1 is a side exploded view of one embodiment of a soft tissue reattachment mechanism.

FIG. 2 is a cross-sectional view of one aspect of the embodiment of FIG. 1, taken along the lines 2-2 and viewed in the direction of the arrows.

FIG. 3 is a side view of another embodiment of part of the structure shown in FIG. 1.

FIG. 4 is a side view of another embodiment of part of the structure shown in FIG. 1.

FIG. 5 is a side view partially in cross-section of the embodiment of part of the structure shown in FIG. 1 with additional structure.

FIG. 6 is a side view partially in cross-section of the embodiments shown in FIG. 5 during implantation.

FIG. 7 is a side view partially in cross-section of the embodiments shown in FIG. 5 during implantation.

FIG. 8 is a side view partially in cross-section of the embodiments shown in FIG. 5 during implantation.

FIG. 9 is a side view partially in cross-section of the embodiments shown in FIG. 5 during implantation.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

Referring now generally to the figures, there is shown embodiments of a non-suture soft tissue attachment device 20, which includes an anchor 22 and a lock 24. Anchor 22 and lock 24 are made of biocompatible materials such as sturdy plastics (e.g. polyetheretherketone or PEEK), metals (e.g. stainless steel or shape memory alloys such as nitinol), or other materials that are capable of performing at least the functions described below. Anchor 22 and lock 24 may also be made of biodegradeable or resorbable materials, so that device 20 can initially hold soft tissue to bone, and as the soft tissue becomes naturally attached to the bone via growth of new tissue, device 20 can degrade or be resorbed. Further, anchor 22 and/or lock 24 may be made of a material that permits ingrowth of tissue such as bone, ligament, tendon, muscle or other tissue.

The illustrated embodiment of anchor 22 is monolithic, i.e. a single piece, and includes a stem portion 26, a head portion 28, and a neck portion 30. Stem 26 is generally linear in this embodiment, and has substantial flexibility and elasticity along its length, allowing bending of stem 26 in any of a variety of directions. Accordingly, where stem 26 is made of harder materials such as metals, it may be relatively thin in order to permit such bending. Head 28 is directly connected to neck 30, which is directly connected to stem 26. As explained further below, neck 30 is flexible and elastic to allow bending, along with repositioning of head 28 with respect to stem 26.

Stem 26, as already noted, is generally linear in this embodiment, having a central longitudinal axis 32. A first end 34 of stem 26 is tapered, for example to a point, to make placement of lock 24 easier. A series of ridges 36 extends along at least a portion of stem 26. In the illustrated embodiment ridges 36 are generally in a row along longitudinal axis 32, and extend along a portion of stem 26 from a point adjoining or adjacent to neck 30 to a point about halfway along stem 26. In other embodiments, ridges 36 may start further down stem 26 from neck 30, for example a distance slightly less than an average or expected thickness of bone and soft tissue at the place anchor 22 is to be implanted, or ridges 36 may be along most or the entirety of stem 26. Ridges 36 of an illustrated embodiment include a first surface 38 generally facing head 28 and a second surface 40 generally facing end 34 of stem 26. Surface 38 is in a plane perpendicular to axis 32, and an acute included angle is between surfaces 38 and 40. As further explained below, surface 38 of a ridge 36 helps prevent sliding of lock 24 toward end 34 of stem 26, while surface 40 allows lock 24 to slide toward head 28 along stem 26.

The illustrated embodiment of stem 26 is substantially square and has ridges 36 on opposed sides. It will be understood from this disclosure that ridges 36 may be placed on all sides, on a single side, or on adjoining sides of stem 26. Similarly, it will be understood that stem 26 may have a substantially circular or oval cross section, a cross section of a regular or irregular polygon, or of other cross section. It is believed that placement of ridges 36 on opposing sides of stem 26, as for example on opposing sides of a rectangle or oval, provides the greatest stability for device 20. Ridges 36 are placed about 2 millimeters apart along stem 26, thus providing steps of 2 millimeters for the movement of lock 24 along stem 26, as further described below. That spacing between ridges 36 is believed to be particularly efficacious since significantly larger gaps could offer a choice between loose hold of the soft tissue to the bone and injurious compression of the soft tissue, and significantly smaller gaps may force reductions in the dimensions of ridges 36 that could limit their effectiveness in blocking lock 24.

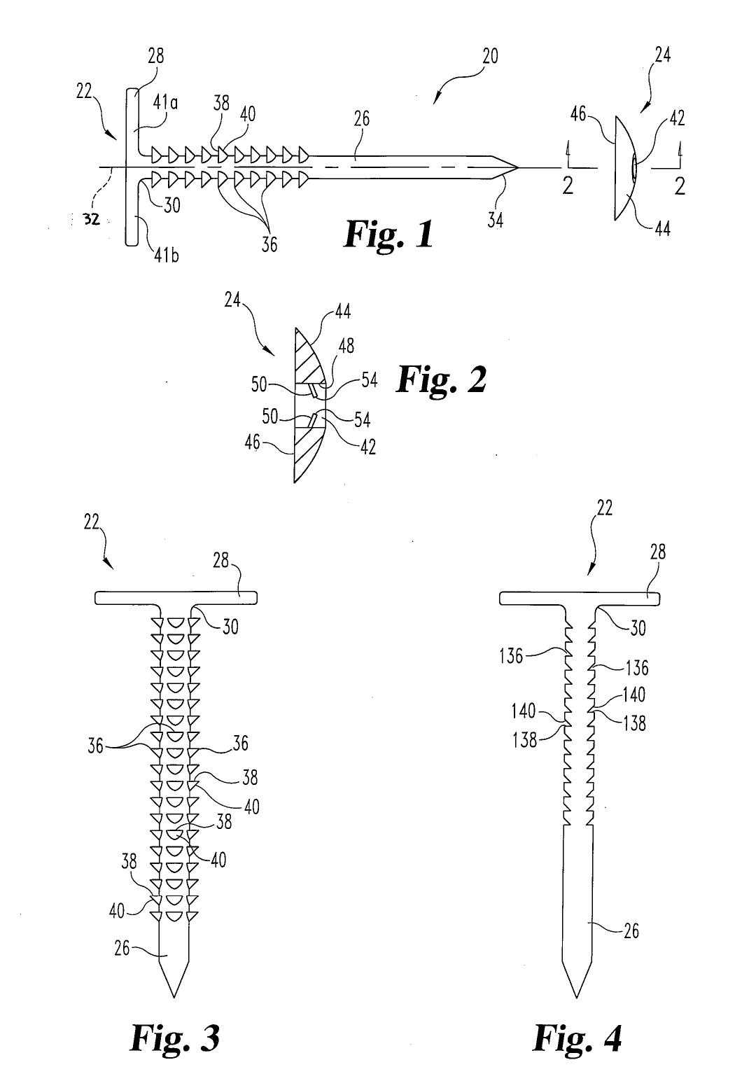

Head 28 is generally planar in the illustrated embodiment and has a thickness that is approximately the same as the diameter or thickness of stem 26. When unstressed, head 28 is at least approximately perpendicular to axis 32 of stem 26. Head 28 includes two substantially straight planar side portions 41a (one of which is shown in FIG. 1 and similar views, and the other is on the diametrically opposed unseen side of head 28 in FIG. 1), and may be substantially rectangular or may have rounded edges between side portions 41a. The distance between side portions 41a is larger than the width or thickness of stem 26 in the illustrated embodiment. A length of head 28, measured substantially perpendicularly to the distance between side portions 41a, is larger than that distance. An underside 41b of head 28 merges into neck 30. Neck 30 is monolithic with and directly connected to both stem 26 and head 28 in this embodiment, and is flexible and elastic so that head 28 can be moved with respect to stem 26 by bending at neck 30, and when such moving or bending stress is relieved, head 28 substantially returns to or toward its unstressed state perpendicular to axis 32 of stem 26. It is preferred that neck 30 is made out of the same material as stem 26 and head 28 of a thickness to provide such flexibility and elasticity. In other embodiments, neck 30 may be made of a different material (e.g. certain rubbers or plastics) and be monolithically joined to stem 26 and head 28, or neck 30 may include a supporting collar of rubber or other biocompatible material to enhance flexibility or elasticity. In other embodiments, neck 30 is not elastic, or not sufficiently elastic to move head 28 to a position substantially perpendicular to stem 26.

Lock 24 is generally in the shape of a circular disk with a hole 42 in the center, in the illustrated embodiment. Lock 24 has an upper surface 44 and a lower surface 46, with upper surface 44 being rounded and lower surface 46 being substantially flat in the illustrated embodiment. Hole 42 extends between surfaces 44 and 46 so that lock 24 is generally in the form of a washer that is thicker in the middle and thinner at the circumference. Hole 42 is defined by an inner wall 48, and has a diameter at least slightly larger than the diameter or thickness of stem 26. The illustrated embodiment includes two extensions or pawls 50 extending from wall 48. Pawls 50 generally point toward surface 44, i.e. the portion 52 of pawl 50 adjoining wall 48 is closer to surface 46 of lock 24 than is a tip portion 54 of pawl 50. Pawls 50 are generally linear in this embodiment, and are of a length such that they can enter the spaces between ridges 36. The distance between tips 54 of pawls 50 is at least slightly smaller than the distance between the outermost portions of ridges 36. As lock 24 is slid onto anchor 22, stem 26 moves through hole 42. When pawls 50 meet ridges 36, each pawl 50 bends at least slightly due to interference with surface 38 of the particular ridge 36, and once past the ridge, pawl 50 elastically snaps back. If it is attempted to pull back on lock 24 to move it toward end 34 of stem 26, then each pawl 50 engages surfaces 38 of respective ridges 36 to inhibit or prevent such movement. It will be understood from this disclosure that a single pawl 50 may be provided in lock 24, particularly if only one set of ridges 36 is on stem 26, or that more than two pawls 50 may be provided, particularly if there are more than two sets of ridges on stem 26. Certain embodiments have the same number of pawls 50 in lock 24 as there are sets of ridges 36 on stem 26, and placed in lock 24 so that each pawl 50 engages a particular set of ridges 36. In other embodiments, multiple pawls 50 may be provided one behind another, so that two or more pawls 50 engage the same set of ridges 36.

A placement tube 60 is also shown in the figures. In the illustrated embodiment, tube 60 has a lumen 62 with a diameter at least slightly larger than the distance between side portions 41a of head 28. Tube 60 also has an outer diameter that is at least slightly larger than hole 42 in lock 24. As will be further explained below, tube 60 can be used in a minimally-invasive or other surgical procedure to help insert anchor 22 in an appropriate location, and to move lock 24 on stem 26 to secure device 20. In the illustrated embodiment, the length of tube 60 is greater than the length from head 28 to end 34 of anchor 22, although in other embodiments the length of anchor 22 may be longer than tube 60, so that end 34 extends from tube 60 for control or manipulation by the surgeon.

Embodiments of the use of device 20 will now be described in the context of a repair of a rotator cuff injury, which is one non-limiting example of a procedure in which device 20 is useful. It will be understood from this disclosure that other uses of device 20 for attachment or reattachment of soft tissue to bone or for other orthopedic tasks will be possible. Further, the context of a minimally-invasive surgical procedure will also be used. It will be understood from this disclosure that the described devices and methods may be used in open surgical procedures as well.

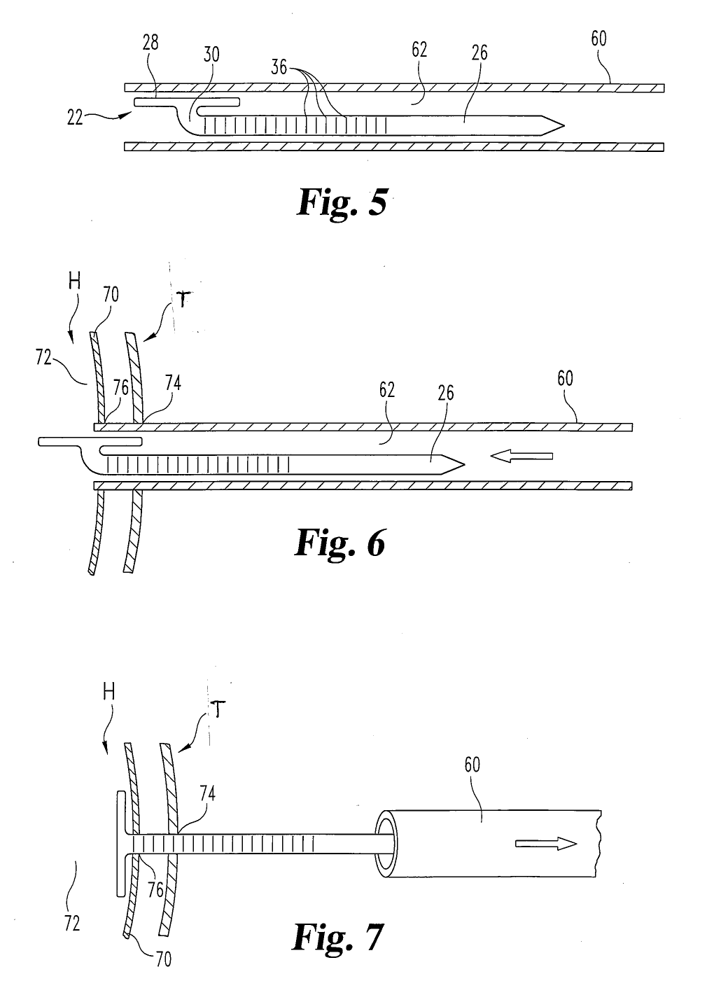

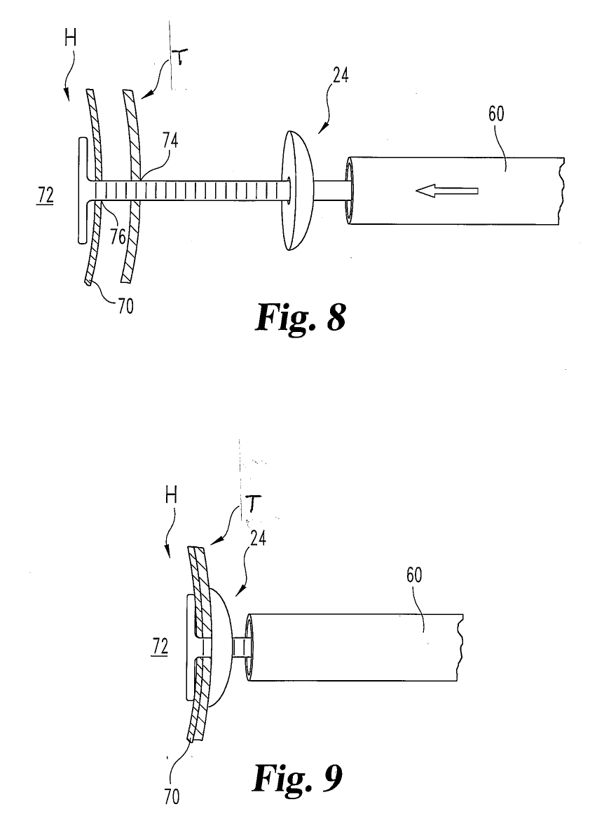

Thus, referring now to FIGS. 5-9, there is shown schematically a portion of a humerus H and an associated portion of a rotator cuff tendon T that has been separated from the head or a tubercle of the humerus. Other soft tissues, such as the coracohumeral ligament or the long head of the biceps brachii muscle, can also be attached to or held with respect to the humerus using device 20. The tissues are shown in cross section, so that the hard cortical bone portion 70 and the softer cancellous, spongy or bloody bone portion 72 of the humerus can be seen. Access to the surgical site is gained minimally-invasively, via a relatively narrow percutaneous approach. Once access to the site is gained, the surgeon arranges the tissue as desired, in a position that will promote healing. For example, the surgeon may position a portion of the rotator cuff tendon over a portion of the head or tubercle of the humerus, so that when attached, the tendon and the remainder of the rotator cuff will be in an approximately normal relationship. In embodiments in which tube 60 includes a point or sharpened end 61, the surgeon may use tube 60 as an awl to pierce the rotator cuff tendon and punch into the humerus to a depth below the cortical bone 70, forming a hole 74 in the ligament and a concentric hole 76 in the humerus. Although a drill could be used to form holes 74 and 76, use of tube 60 as an awl reduces the number of steps and amount of equipment required. If a drill is used, tube 60 may be placed through hole 74 and into hole 76, either over the drill (not shown) or following the drill's removal from the site. If tube 60 is inserted over a drill, then the drill is withdrawn from inside tube 60 once tube 60 is positioned in hole 76 in the humerus.

Anchor 22 is then inserted through lumen 62 of tube 60. Neck 30 of anchor 22 is bent so that head 28 is adjacent to and approximately parallel to stem 26. In that configuration, anchor 22 can be inserted into tube 60 and guided through lumen 62 toward the humerus. The surgeon may insert anchor 22 by hand, or using an appropriate gripping instrument to hold anchor 22 and move it through tube 60. When head 28 arrives at the distal end of tube 60, the surgeon pushes it out of tube 60, through hole 76 in cortical bone 70 and into the spongy cancellous bone 72 of the humerus. When head 28 is free of tube 60, the elasticity of neck 30 causes head 28 to substantially resume its initial configuration, i.e. generally perpendicular to stem 26. With that resumption of an unstressed configuration, the lateral dimension of head 28 is larger than the hole 76 in cortical bone 70 of the humerus H. Cancellous bone 72 can be displaced by head 28, but head 28 is too large to be withdrawn through the cortical bone 70, and is thus anchored in the humerus. The surgeon can then withdraw tube 60 from around stem 26, and may give stem 26 a pull to force surface 41b of head 28 against the interior of cortical bone 70 of the humerus.

With tube 60 withdrawn, the surgeon may access tip 34 of stem 26, which may be within the patient or extending through a port or other device to a point outside the skin. Lock 24 is then maneuvered to tip 34, and stem 26 is inserted into hole 42 of lock 24. With the illustrated embodiment, lock 24 can be moved without any interference along the smooth part of stem 26. As ridges 36 are inserted into hole 42, they engage pawls 50. With ridges 36 and pawls 50 oriented as described above, as lock 24 is advanced further on stem 26, pawls 50 are bent by ridges 36, and a particular pawl 50 snaps back once a particular ridge 36 is cleared. Lock 24 cannot be pulled backward at this point, due to interference between pawls 50 and perpendicular surfaces 38 of ridges 36.

Lock 24 may be advanced with the surgeon's fingers in certain embodiments, but in some minimally-invasive procedures tube 60 or another tool can be used to push lock 24. Thus, once lock 24 is on stem 26, tube 60 may be inserted over stem 26 and against upper surface 44 of lock 24. Advancing tube 60 pushes lock 24 along stem 24 and over ridges 36. Whether tube 60 or another device is used to push lock 24 or not, lock 24 is advanced until it at least abuts the rotator cuff tendon. In most embodiments, lock 24 will be advanced until it presses the tendon firmly against the humerus, anchoring the tendon and the humerus together. The cortical portion of the humerus and the rotator cuff tendon (in this example) are squeezed between head 28 and lock 24. Once lock 24 is advanced to the position desired by the surgeon, a portion of stem 26 extending above surface 44 of lock 24 can be cut off. Once the excess stem 26 and the tube 60 (and a port device, if used) is removed, the minimally-invasive incision can be closed with sutures, staples or by other elements or substances.

With device 20 so placed, scar or other tissue can grow around device 20 and between or around the humerus and ligament, so that the ligament and humerus are reattached. In embodiments in which device 20 is not biodegradeable, it may remain in the body indefinitely. In embodiments in which device 20 is made of a material that allows tissue ingrowth, such scar or other tissue will grow around and through device 20. In embodiments in which device 20 is biodegradeable or resorbable, the material is chosen so that the holding power of device 20 does not dissipate before such scar or other tissue has grown and can maintain the joinder of the ligament and the humerus.

Device 20 provides an exact and firm attachment of soft tissue to bone, and eliminates many of the problems surrounding suture-based current methods of rotator cuff repairs and similar surgeries. The open procedure needed for current suture repairs is not needed with device 20, enabling less trauma to the tissues and quicker average recovery periods with less pain. The high skill required for tying sutures to precisely the right size for holding soft tissue to bone is not required in using device 20, and thus reattachment or other repair procedures are made much easier. Even where sutures are accurately tied, slippage or other problems with the knot or the suture can make follow-up surgery necessary. Device 20 provides a certain hold, with force applied precisely where and in the amount the surgeon desires, overcoming those drawbacks of sutures. A number of mechanical devices to hold sutures, or thermal or other treatments to meld or otherwise link sutures together, have been proposed, but the devices disclosed herein take the different approach of doing away with sutures as a means of holding together bone and soft tissue altogether.

The above description uses the term “ridges” to describe the irregular surface along part of stem 26. Ridges 36 thus may be thought of as surfaces 38 and 40 rising above a base surface of stem 26. It is also possible to fashion a series of grooves 136 that extend below a base surface of stem 126, shown in one embodiment in FIG. 4. Stem 126 is essentially the same as stem 26, and all of the description applicable to stem 26 may be applied to stem 126, so that stem 126 can be a part of an embodiment of device 20. Grooves 136 of stem 126 are formed by two surfaces 138 and 140. Surface 138 is substantially perpendicular to axis 32 of stem 126, and surface 140 is oblique to axis 32 and generally points toward head 28 (i.e. as the surface extends away from axis 32, it extends toward head 28). Operation of stem 126 with lock 24 is essentially the same as described above with respect to stem 26 and lock 24.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. An apparatus for attaching soft tissue to bone, comprising:
an anchor having a head flexibly joined to a stem, said head being pivotable with respect to said stem so that in a first relative position said head is substantially parallel to said stem and in a second relative position said head is transverse to said stem, said head having a lower surface facing said stem;
a locking member separate from said head, said locking member having a central opening adapted to receive said stem, and a surface that faces said head when said aperture receives said stem, said locking member adapted to remain in contact with said anchor so that the tissue is held between said head surface and said locking member surface.

2. The apparatus of claim 1, wherein said head has two substantially planar side portions and a width between said side portions, and further comprising a tube with a lumen having an inner diameter larger than said width, wherein said anchor can be passed through said lumen.

3. The apparatus of claim 2, wherein said head has a length transverse to said width, said length being larger than said inner diameter.

4. The apparatus of claim 2, wherein said tube has a sharpened end adapted to pierce tissue.

5. The apparatus of claim 1, wherein said stem has a plurality of ridges and said locking member has at leas one pawl extending into said aperture, said at least one pawl cooperating with said ridges so that said locking member is held on said stem.

6. The apparatus of claim 1, wherein said stem has a plurality of grooves and said locking member has at leas one pawl extending into said aperture, said at least one pawl cooperating with said grooves so that said locking member is held on said stem.

7. The apparatus of claim 1, wherein at least one of said anchor and said locking member include a material that permits tissue ingrowth.

8. The apparatus of claim 1, wherein at least one of said anchor and said locking member include a resorbable or biodegradeable material.

9. A non-suture apparatus for attaching soft tissue to bone, comprising:
an anchor, said anchor having a monolithic head, stem, and neck,
said head being substantially inflexible and having an underside that is directly connected to said neck,
said stem being elongated with a central axis, a first end that is directly connected to said neck and a second opposing end, said second end being tapered, said stem having a series of ridges that are substantially perpendicular and offset from said central axis, said ridges being found at least on a portion of said stem adjacent to said neck,
said neck having at least a portion that is flexible and elastic, wherein said anchor has a first unstressed configuration in which said head is transverse to said stem, and said flexible and elastic neck is bendable so that said head can be subjected to stress and moved to a second configuration in which said head is substantially parallel to said stem, said elasticity of said neck allowing said anchor to change to substantially said first configuration on the removal of the stress;
a lock placed on said stem, said lock having an upper surface generally facing away from said head, a lower surface generally facing toward said head, and a central hole between said surfaces defined by an internal wall, said central hole having a width larger than a width of said stem, said lock including at least one pawl that extends from said internal wall in a direction generally toward said upper surface, said at least one pawl being of a length to be able to interengage with said ridges on said stem, so that said lock can easily slide over said ridges on said stem toward said head, and said at least one pawl prevents said lock sliding over said ridges on said stem away from said head.

10. A method of minimally-invasively performing rotator cuff repair surgery, comprising:
creating minimally-invasive holes through detached soft tissue and through the cortical bone of the humerus so that said holes align when the soft tissue is placed on the humerus in a desired relationship;
inserting a narrow tube through said holes;
placing a monolithic anchor through said tube and said holes, said anchor having a stem with ridges, a head having a width larger than a width of said holes, and a flexible and elastic neck directly joining said stem and head, said neck being bent so that said head is substantially parallel to a portion of said stem when said anchor is within said tube, and wherein said elasticity of said neck causes said head to move with respect to said stem when said head has cleared said hole in the humerus, so that said head is transverse to said stem and abuts an cortical bone of the humerus from inside the humerus, preventing said anchor from being pulled through said holes;
placing a lock on said stem, said lock having a central hole and at least one pawl configured to interengage with said ridges of said stem so that said lock can be moved toward said head over said ridges, and that said lock cannot be moved away from said head over said ridges due to interaction between said at least one pawl and one or more of said ridges; and
moving said lock along said stem so that the soft tissue and the cortical bone of the humerus are pressed between said head and said lock.
